(19) United States
(12) Patent Application Publication
(10) Pub. No.: US 2010/0240091 A1
(43) Pub. Date: Sep. 23, 2010

(54) BIOCHEMICAL TEST FOR CONFIRMING THE PRESENCE OF L. MONOCYTOGENES

(75) Inventors: Bernadette Blanc, Dardilly (FR); Daniel Monget, Saint Sorlin en Bugey (FR); Nadine Perrot, Montluel (FR)

Correspondence Address: OLIFF & BERRIDGE, PLC, P.O. BOX 320850, ALEXANDRIA, VA, 22320-4850, US

(73) Assignee: BIOMERIEUX, MARCY L'ETOILE (FR)

(21) Appl. No.: 12/681,014

(22) PCT Filed: Oct. 29, 2008
(86) PCT No.: PCT/FR08/51946

§ 371 Date: Mar. 31, 2010

(30) Foreign Application Priority Data
Oct. 30, 2007 (FR) 0758666

(51) Int. Cl. C12Q 1/04

(52) U.S. Cl. 435/34

(57) ABSTRACT

The invention relates to a biochemical test for confirming the presence of L. monocytogenes, characterized in that it comprises at least one phosphatidylinositol phospholipase C (PI-PLC) substrate and at least one alpha-mannosidase substrate.

The present invention relates to the identification of pathogenic bacteria of the Listeria genus, and more specifically the species Listeria monocytogenes.

The isolation and identification of the Listeria monocytogenes bacterium is a major problem in monitoring food-processing hygiene and medical bacteriology. Among the bacteria of the Listeria genus, only the Listeria monocytogenes species is known to be pathogenic to humans. It can cause listeriosis, sometimes fatal (25 to 30% of cases) in immunodepressed individuals, infants or pregnant women. The other species of Listeria are not pathogenic or are only pathogenic to animals. This is in particular the case of Listeria ivanovii.

Even though the risks of human listeriosis have regressed in most developed countries over the past few decades, modern society demands more and more safety which, while it can accept sporadic cases, cannot tolerate epidemics.

In the context of diagnosing bacteria conditions in humans, it is therefore important to clearly distinguish Listeria monocytogenes from the other species of the Listeria sp. genus which are not pathogenic.

The Listeria sp genus today comprises six species, of which only Listeria monocytogenes is pathogenic to humans.

Microorganisms are responsible for serious epidemics in humans, following contaminations of food origin. Two species of Listeria are mainly isolated in foods (milk and milk products essentially), these species being: nonpathogenic Listeria innocua and pathogenic Listeria monocytogenes.

These two species have many common biochemical characteristics and it is consequently difficult to differentiate them. The beta-hemolysis test, for example, is based on determining a beta-hemolytic activity linked to the production, by the bacterium, of a substance which causes the lysis of red blood cells (listeriolysin). This test is performed on sheep or horse blood agar, but the response is often light. This test is not therefore very reliable and, while it makes it possible to differentiate the two species mainly encountered, namely L. monocytogenes (positive response) from L. innocua (negative response), it is not specific for the pathogenic species.

The CAMP test is another test, which is associated with the beta-hemolysis test previously described. The test is carried out on trypticase soy agar containing 5% of washed sheep red blood cells. The beta-hemolytic S. aureus strain CIP 5710 is inoculated in a streak perpendicular to the streaks formed by the culture of the Listeria strain to be tested. The reinforcement of the staphylococcal hemolysis at the contact of the two zones indicates a positive reaction. In practice, the CAMP test is a laborious technique to implement, and, like the hemolysis, is not always very reliable.

It is also possible to use culture media or identification media. In this respect, patent EP496680, granted to the applicant, describes a bacteriological analytical process for differentiating the species Listeria monocytogenes from the other bacteria of the Listeria sp genus. According to this process, a reaction medium comprising a chromogenic or fluorogenic substrate capable of being hydrolyzed by an enzyme called glycine aminopeptidase is used. This very advantageous technique has a drawback which lies in the fact that the Listeria monocytogenes species is the only one which does not have any glycine aminopeptidase enzymatic activity. The detection of Listeria monocytogenes is carried out by means of a negative activity, which is not easy, the use of a negative test lacking specificity in the case of a mutant or if the other species are stressed and do not respond with a normal activity.

It is possible to differentiate the Listeria monocytogenes and Listeria ivanovii species from the other Listeria by demonstrating phosphatidylinositol-specific phospholipase C (PI-PLC) activity. This is because it has been demonstrated that PI-PLC is secreted into the culture medium by certain species of the Listeria genus, such as Listeria monocytogenes and Listeria ivanovii (Leimeister-Wächter et al., Mol. Microbiol. (1991) 5(2), pp. 361-366). In this respect, the Ottaviani Agosti Agar medium is a chromogenic medium which uses, on the one hand, a β-glucosidase substrate which allows the detection of all the species of the Listeria genus and, on the other hand, the demonstration of the PI-PLC activity of Listeria monocytogenes and Listeria ivanovii through the appearance of a halo around the colony.

Whatever the technique used, it is necessary to systematically carry out a test for confirming Listeria monocytogenes, generally using a second culture medium, for which a 24 h incubation step is necessary. In this respect, mention may be made of the ALOA Confirmation (AES), or CHROMagar Identification listeria (BBL) confirmation tests. All in all, a period of 48 h is therefore necessary to identify and confirm the presence of Listeria monocytogenes: use of a first culture medium, which requires 24 h of incubation, and then the second step of confirmation, which also requires an incubation of 24 h. This 48 h period is long, and it is desirable to shorten this period in order to propose a reliable diagnosis as rapidly as possible.

The objective of the present invention is to propose a method of detection which allows the differentiation of the Listeria monocytogenes species with respect to all the other species of Listeria spp. In particular, the invention proposes a novel confirmation test, which is a very rapid biochemical test since it can be carried out in less than 6 h.

Before proceeding with the description of the invention, the definitions below are given in order to facilitate the disclosure of the invention.

The term biochemical test is intended to mean a test for carrying out a biological reaction demonstrating the presence of bacteria. Such a test can in particular be implemented directly using an inoculum, without carrying out a growth step. It is then the preformed enzymes provided by the bacterial suspension which are detected.

Such tests are in particular used in the API® range or the Vitek 2 system. These tests packaged in dehydrated form are brought into contact, under incubation, with the sample to be analyzed. The biochemical reaction sought is detected by means of a change in state of the test (appearance or disappearance of a coloration, of a fluorescence). The test is then read visually or by means of a reader, directly or after the addition of a developing reagent.

The API® range is based on a probability method of numerical identification, combining a strip of biochemical tests, in cupules, and a database. Judicious combination of the tests provides the characterization of a microorganism group and distinction between the species.

The VITEK 2 system is an entirely automated identification and antibiogram system which uses a 64-microwell card. By eliminating the manual steps, the automation makes it possible to save time in the laboratory and guarantees uniform analytical procedures, thereby improving the reliability of the results. The identification carried out on this system uses a probability method similar to that of the API strips. The automation of the reading enables kinetic reading of the biochemical tests carried out and thus enables an identification in 4 to 8 hours for the common Gram-positive microorganisms and also for Listeria monocytogenes.

The biochemical test according to the invention is a test for confirming the presence of L. monocytogenes, comprising a PI-PLC substrate and an α-mannosidase substrate, but may also comprise mineral salts, peptones, carbohydrates, one or more compounds for limiting pH variations, one or more enzyme inducers.

The term substrate is intended to mean any molecule capable of directly or indirectly generating a detectable signal due to an enzymatic or metabolic activity of the microorganism.

The substrate may in particular be an enzyme substrate, i.e. a substrate that can be hydrolyzed by an enzyme so as to give a product allowing the direct or indirect detection of a microorganism.

For the purpose of the present invention, this substrate is in particular a phosphatidylinositol phospholipase C (PI-PLC) substrate or an α-mannosidase substrate.

This substrate may in particular comprise a first portion specific for the enzymatic activity to be revealed and a second portion which acts as a label, hereinafter referred to as label portion. This label portion may be chromogenic, fluorogenic, luminescent, etc.; preferably, the substrates used in the present invention are chromogenic. Mention may in particular be made of substrates based on indoxyl and derivatives thereof, which are particularly suitable in the case of phosphatidylinositol phospholipase C (PI-PLC) substrates, substrates based on nitrophenol and derivatives, which are particularly suitable in the case of α-mannosidase substrates, but also substrates based on hydroxyquinoline or on esculetin or on alizarin or on catechol or on aminophenol and on naphthol and their derivatives. For the fluorogenic labels, mention may be made of derivatives of coumarins and in particular of umbelliferone, of naphthol, of resorufin or of fluorescein.

According to the present invention, the substrate is preferably chosen from substrates based:
on indoxyl (3-indoxyl, 5-bromo-3-indoxyl, 4-chloro-3-indoxyl, 5-iodo-3-indoxyl, 5-bromo-4-chloro-3-indoxyl, 5-bromo-6-chloro-3-indoxyl, 6-bromo-3-indoxyl, 6-chloro-3-indoxyl, 6-fluoro-3-indoxyl, 4,6-dichloro-3-indoxyl, 6,7-dichloro-3-indoxyl, 4,6,7-trichloro-3-indoxyl, 5-bromo-4-chloro-N-methyl-3-indoxyl, N-methyl-3-indoxyl, etc.);
on nitrophenol (ortho-nitrophenol, para-nitrophenol, etc.).

Preferably, the PI-PLC substrate is a chromogenic substrate. Preferably, the PI-PLC substrate is an indolyl-phosphatidyl-myo-inositol, preferably 5-bromo-4-chloro-3-indoxyl myoinositol phosphate (X-indolyl-phosphatidyl-myo-inositol).

Preferably, the α-mannosidase substrate is a chromogenic or fluorogenic substrate. Preferably, the α-mannosidase substrate is a chromogenic substrate. Preferably, the α-mannosidase substrate is a mannopyranosidase substrate. Preferably, the mannopyranosidase substrate is 4-nitrophenyl-α-D-mannopyranoside.

Preferably, the concentration of PI-PLC substrate is between 5 and 0.1 g/l, preferably between 1.5 and 0.5 g/l.

Preferably, the concentration of α-mannosidase substrate is between 2 and 0.1 g/l, preferably between 1 and 0.5 g/l.

The term reaction medium is intended to mean a medium comprising all the elements necessary for the expression of a metabolism and/or for the growth of microorganisms. The reaction medium may be solid, semi-solid or liquid. The term “solid medium” is intended to mean, for example, a gelled medium. Agar is the conventional gelling agent in microbiology for culturing microorganisms, but it is possible to use gelatin or agarose. A certain number of preparations are commercially available, for instance Columbia agar, Trypticase-soy agar, MacConkey agar, Sabouraud agar or, more generally, those described in the Handbook of Microbiological Media (CRC Press).

The reaction medium may comprise one or more elements in combination, such as amino acids, peptones, carbohydrates, nucleotides, minerals, vitamins, active molecules such as antibiotics, enzymes, surfactants, buffers, phosphate salts, ammonium salts, sodium salts, metal salts, one or more substrates for detecting an enzymatic or metabolic activity, etc.

The medium may also comprise a colorant. By way of indication, mention may be made, as colorant, of Evans blue, neutral red, sheep blood, horse blood, an opacifier such as titanium oxide, nitroaniline, malachite green, brilliant green, etc.

For detecting the Listeria genus, mention may in particular be made of the Paleam and Oxford media, conventional selective media commonly used industrially and mentioned in the international standards ISO 11290-1 and 11290-2 for investigating and counting Listeria monocytogenes, or chromogenic media such as the OAA medium, which, in addition to detection, enables the presumptive identification of L. monocytogenes.

The term biological sample is intended to mean a clinical sample, derived from a biological fluid specimen, or a food sample, derived from any type of food. This sample may thus be liquid or solid and mention may be made, without limitation, of a clinical sample of blood, cerebrospinal fluid or placenta, a food sample from water or from drinks such as milk or a fruit juice; from yoghurt, from meat, from eggs, from vegetables, from mayonnaise, from cheese; from fish, or a food sample derived from an animal feed.

In this respect, the invention relates to a biochemical test for confirming the presence of L. monocytogenes, characterized in that it comprises at least one phosphatidylinositol phospholipase C (PI-PLC) substrate and at least one α-mannosidase substrate.

According to one preferred embodiment of the invention, the PI-PLC substrate is an indolyl-phosphatidyl-myo-inositol, preferably 5-bromo-4-chloro-3-indoxyl myoinositol phosphate.

According to one preferred embodiment of the invention, the alpha-mannosidase substrate is a mannopyranosidase substrate, preferably 4-nitrophenyl-α-D-mannopyranoside.

According to one preferred embodiment of the invention, the test also comprises a PI-PLC activator or activators, such as, in particular, magnesium alpha-glycerophosphate or bovine serum albumin.

According to one preferred embodiment of the invention, the concentration of PI-PLC substrate is between 5 and 0.1 g/l, preferably between 1.5 and 0.5 g/l.

According to one preferred embodiment of the invention, the concentration of α-mannosidase substrate is between 2 and 0.1 g/l, preferably between 1 and 0.5 g/l.

The invention also relates to the use of a biochemical test as described above, i.e. a biochemical confirmation test comprising a phosphatidylinositol phospholipase C (PI-PLC) substrate and at least one alpha-mannosidase substrate, as defined above, for confirming the presence of L. monocytogenes.

The test according to the invention is particularly suitable for use as a single unit or use combined with a series of biochemical tests so as to give rise to an identification device in the API® range or Vitek® system. Currently, the identification of L. monocytogenes on an API strip or Vitek system uses a reaction profile established by the linking together in a series of the positive or negative results of several unitary biochemical tests. The test according to the invention can advantageously be integrated into such devices so as to reduce the number of tests.

The invention also relates to a method for identifying Listeria monocytogenes, characterized in that it comprises the following steps:
a) providing a reaction medium for detecting the Listeria genus,
b) inoculating the medium with a biological sample to be tested,
c) leaving to incubate,
d) revealing the presence of the Listeria genus,
e) confirming the presence of L. monocytogenes by means of a biochemical test according to the invention, comprising a phosphatidylinositol phospholipase C (PI-PLC) substrate and at least one alpha-mannosidase substrate, as defined above.

The inoculation of the microorganisms can be carried out by any of the inoculation techniques known to those skilled in the art. An incubation step can be carried out at a temperature for which the enzymatic activity that it is desired to detect is optimal, it being possible for those skilled in the art to easily select said temperature according to the enzymatic activity to be detected. Step d) can be carried out by visual examination or by colorimetry or fluorimetry.

For detecting the Listeria genus, mention may in particular be made of the OAA medium (bioMerieux, ref 43641) which makes it possible to detect the species of the Listeria genus by blue coloration of the colonies (beta-glucosidase activity).

The invention also relates to a method for identifying Listeria monocytogenes, characterized in that it comprises the following steps:
a) providing a biochemical test or a combination of biochemical tests for detecting the Listeria genus,
b) revealing the presence of the Listeria genus,
c) confirming the presence of L. monocytogenes by means of a biochemical test according to the invention, comprising a phosphatidylinositol phospholipase C (PI-PLC) substrate and at least one alpha-mannosidase substrate, as defined above.

For detecting the Listeria genus, mention may in particular be made of the morphology and the Gram reaction, with which one commonly associates the test with esculin, catalase and tests for fermentation of sugars such as xylose, rhamnose, mannitol, ribose, or α-methyl-D-mannoside.

The examples below are given by way of explanation and are in no way limiting in nature. They will make it possible to understand the invention more clearly.

### EXAMPLE 1

### A) Biochemical Test According to the Invention

A biochemical test according to the invention comprises the following elements in g/l:

| | |
|---|---|
| Tris buffer | 37.5 |
| Peptone extract | 2.5 |
| Yeast extract | 0.25 |
| Mg alpha-glycerophosphate | 1.25 |
| Bovine serum albumin | 7.5 |
| X-myo-inositol-1-phosphate | 2 |
| 4-Nitrophenyl-α-D-mannopyranoside | 2.25 |

Final pH=7.

After 20 μl of solution have been deposited in a plastic support, the test was dehydrated at a temperature of 40° C. for 24 h.

The test was evaluated for obtaining a reaction in 4 to 6 h at 37° C.+1-2° C. after rehydration with 50 μl of demineralized water and addition of a colony, or after having been rehydrated with 50 μl of a bacterial suspension having an opacity equivalent to 0.5 McF.

### B) Reading and Interpretation of the Test

After 4 and 6 h of incubation of the test at 37° C.+/−1° C., the coloration of the test is read and interpreted according to the following grid:

| Coloration | interpretation | presumptive identification |
|---|---|---|
| colorless | negative | Listeria sp * |
| yellow | negative | |
| turquoise blue | negative | |
| green | positive | L. monocytogenes |

### C) Validation of the Test According to the Invention

The test according to the invention was evaluated, initially, using an inoculum of 0.5 McFarland on 90 pure strains of the Listeria genus cultured beforehand on OAA medium (Ref 43641). The 6 species of the Listeria sp genus were divided up in the following way: L. monocytogenes (30 strains), L. ivanovii (20) (comprising 10 strains of the subspecies L. ivanovii spp londoniensis and L. ivanovii spp ivanovii), L. grayii (10), L. seeligeri (10), L. welshimeri (10), L. innocua (10). From 4 h onward, 80% of the L. monocytogenes strains exhibiting a halo on OAA medium (25/25) showed a positive green coloration. After 6 h of incubation, all these strains exhibited the expected green color. After 24 h, the reaction is stable and remains so up to 72 hours of incubation at 37±0.2° C. None of the 60 strains belonging to the species other than L. monocytogenes gave a green coloration. The test therefore exhibits 100% sensitivity in 6 h and 100% specificity within the Listeria sp genus.

The effectiveness of the test according to the invention was also evaluated for an inoculum of a colony from pure strains or from strains derived from food matrices. In the latter case, the matrices were studied after preenrichment in ½ Fraser broth as recommended by the AFNOR (BIO 12-14) short protocol. This step allows regeneration of the bacterial strains which may have undergone stress linked to the treatment of the matrix.

Study on Pure Strains

Fifty L. monocytogenes strains cultured for 24 h at 35° C.±2° C. on OAA medium were inoculated in a proportion of one colony in the biochemical test according to the invention rehydrated with 50 μl of sterile water. The test incubated at 35° C.±2 was positive in 4 h for 94% of the strains which exhibited a characteristic colony on OAA medium (blue colony with halo). After 6 hours, all the L. monocytogenes strains were detected positive (green) on the biochemical test according to the invention.

Study on Matrix

The search for L. monocytogenes on 22 food matrices was carried out according to the AFNOR (BIO 12-14) short protocol, i.e.: 25 g of matrix/stomacher, enrichment in ½ Fraser (24 h-30° C.). After this enrichment phase, 0.1 ml of ½ Fraser broth is inoculated onto OAA medium. The medium is then incubated for 24 h at 35±2° C. Each of the media is verified and when a presumed characteristic L. monocytogenes colony (blue with halo) is observed, said colony is sampled for inoculation of the test according to the invention. Said test is re-hydrated beforehand with 50 μl of medium suspension (70 700).

Among the 22 matrices tested, 11 exhibited a bacterial growth on OAA medium after 24 h. Six exhibited colonies characteristic of the L. monocytogenes species (blue with halo).

The test according to the invention confirmed that the 6 presumed strains belonged to the L. monocytogenes species in 4 h. The identification of the 6 strains as the L. monocytogenes species was confirmed by other identification tools (Listeria API strip, Vitek GP cards and by 16S sequencing).

The 5 other matrices were contaminated with Listeria spp other than L. monocytogenes or other microorganisms. In all these cases, the tests according to the invention carried out on one colony of each of these matrices gave a negative response.

### EXAMPLE 2

The test according to the invention was also validated on 250 pure strains tested with the following breakdown:
L. monocytogenes, 150 strains of varied serotypes and origins.
Listeria sp 50 strains: L. grayii 1, L. innocua 11, L. ivanovii 26 (including L. ivanovii spp ivanovii 6, L. ivanovii spp londoniensis 7), L. seeligeri 5, L. welshimeri 7.
Other genera, 50 strains: Bacillus, Lactobacillus, staphylococci, enterococci.

The strains used are either strains from international collections or strains isolated from food matrices or from the environment. The identification of these strains was established by the laboratory prior to the study, and is considered to be the reference identification. The Listeria strains were identified by API Listeria identification strip.

### Study on Pure Strains

The results obtained on pure strains are given below.

| | | | |
|---|---|---|---|
| | | | 72 h |
| | | | (including |
| 4 h | 6 h | 24 h | 48 h AT) |

| | + | − | + | − | + | − | + | − |
|---|---|---|---|---|---|---|---|---|
| L. monocytogenes | 148 | 2 | 149 | 1 | 149 | 1 | 149 | 1 |
| Listeria ivanovii | 0 | NA | 0 | 26 | 0 | 26 | 0 | 26 |
| Listeria sp and other | 0 | NA | 0 | 36 | 0 | 36 | 0 | 36 |
| genera | | | | | | | | |

Using the OAA agars incubated for 24 h at 37° C., 149 Listeria monocytogenes strains, out of 150 tested, are confirmed by the test according to the invention from 6 h onward. The prolonging of the incubation up to 24 h does not modify the result.

The 50 Listeria sp strains developed from OAA medium.

Only the 26 L. ivanovii strains exhibited characteristic colonies on OAA agar. All these isolates tested using the test according to the invention gave a negative result in 6 h and 24 h.

Among the 50 other-genera strains tested, 12 developed on OAA medium without exhibiting characteristic colonies. The test according to the invention carried out on these isolates is negative.

Reading of the test after 4 h of incubation made it possible to detect 148 isolates. It is therefore possible, with the test according to the invention, to render a positive result (green) from 4 h onward. A negative result can be rendered from 6 h onward. The results were unchanged after 48 h of culture in OAA medium.

Comparable results were obtained on culture media other than OAA (TSA (trypticase soy agar) and blood agar media). The test according to the invention made it possible to confirm the presence of L. monocytogenes at 6 h.

1. A biochemical test for confirming the presence of L. monocytogenes, comprising at least one phosphatidylinositol phospholipase C (PI-PLC) substrate and at least one α-mannosidase substrate.

2. The biochemical test as claimed in claim 1, wherein the PI-PLC substrate is an indolyl-phosphatidyl-myo-inositol.

3. The biochemical test as claimed in claim 2, wherein the indolyl-phosphatidyl-myo-inositol is 5-bromo-4-chloro-3-indoxyl myoinositol phosphate.

4. The biochemical test as claimed in claim 1, wherein the concentration of PI-PLC substrate is between 5 and 0.1 g/l.

5. The biochemical test as claimed in claim 1, α-mannosidase substrate is a mannopyranosidase substrate.

6. The biochemical test as claimed in claim 5, wherein the mannopyranosidase substrate is p-nitrophenyl mannopyranoside.

7. The biochemical test as claimed in claim 1, according to which the concentration of the α-mannosidase substrate is between 2 and 0.1 g/l, preferably between 1 and 0.5 g/l.

8. A process for confirming the presence of L. monocytogenes comprising contacting the biochemical test of claim 1 with a biological sample.

9. A method for identifying Listeria monocytogenes, comprising the following steps:
a) providing a reaction medium for detecting the Listeria genus,
b) inoculating the medium with a biological sample to be tested,
c) leaving to incubate,
d) revealing the presence of the Listeria genus,
e) confirming the presence of Listeria monocytogenes by means of a biochemical test as claimed in claim 1.

10. method for identifying Listeria monocytogenes, comprising the following steps:
a) providing a biochemical test or a combination of biochemical tests for detecting the Listeria genus,
b) revealing the presence of the Listeria genus,
c) confirming the presence of Listeria monocytogenes by means of a biochemical test as claimed in claim 1.
